(19) United States
(12) Patent Application Publication
(10) Pub. No.: US 2004/0073069 A1
(43) Pub. Date: Apr. 15, 2004

(54) Method for processing polyether alcohols

(76) Inventors: Wolfgang Heider, Neustadt (DE); Edward Bohres, Ludwigshafen (DE); Georg Heinrich Grosch, Bad Durkheim (DE); Hartwig Voss, Frankenthal (DE); Gerd Hoppner, Schwarzheide (DE); Kathrin Harre, Dresden (DE); Els Paredis, Ranst (BE); Jurgen Winkler, Schwarzheide (DE); Michael Stosser, Neuhofen (DE); Wilfried Sager, Mutterstadt (DE); Thomas Barth, Ludwigshafen (DE); Hannelore Barth, Heidelberg (DE); Georg Hans Barth, Heidelberg (DE)

Correspondence Address: Basf Corporation, Patent Department, 1609 Biddle Avenue, Wyandotte, MI, 48192, US

(21) Appl. No.: 10/432,474

(22) PCT Filed: Nov. 22, 2001
(86) PCT No.: PCT/EP01/13619

(30) Foreign Application Priority Data
Nov. 22, 2000 (DE) 100 57 891.8
Nov. 22, 2000 (DE) 100 57 892.6

(51) Int. Cl. C07C 41/03

(52) U.S. Cl. 568/672

(57) ABSTRACT

In a process for working up polyether monools which have been prepared by ring-opening polymerization of epoxides by means of OH-functional starters using at least one heterogeneous catalyst, the heterogeneous catalyst or catalysts is/are separated off from the polyether monool by means of a membrane separation process.

The present invention relates to a process for working up polyether alcohols (polyetherols) which have been prepared by ring-opening polymerization of epoxides by means of OH-functional starters using a heterogeneous catalyst, where the heterogeneous catalyst is separated off from the polyether alcohol by means of a membrane separation process, and also relates to a process for preparing such polyether alcohols which comprises a corresponding work-up step. In addition, the present invention relates to the use of the polyether alcohols obtained in this way in the automobile industry in fuel compositions, as fuel additive, in brake fluids, in polyurethanes, especially flexible foams and also as tenside or solvent.

The preparation of polyether alcohols and their work-up are known.

Thus, a widely used process for preparing polyether alcohols comprises carrying out the ring-opening polymerization in the presence of soluble basic catalysts such as sodium hydroxide, potassium hydroxide or cesium hydroxide. These homogeneous basic catalysts generally have to be removed from the polyether alcohols after it has been prepared, since they interfere in its further use. For the removal of such soluble basic catalysts, the prior art describes, for example, the precipitation of the alkali metal ions as phosphates, chlorides or carbonates with the alkali metal salt subsequently being separated off, and treatment of the product mixture obtained with inorganic or organic cation exchangers. On this subject, reference may be made to U.S. Pat. No. 4,306,943, EP-A 0 102 508 (phosphates) and DE-A 43 36 923 (carbonates).

Apart from soluble basic catalysts, insoluble basic catalysts such as magnesium hydroxide or hydrotalcite are also used in the prior art for the ring-opening polymerization of epoxides for preparing polyether monools and poyether polyols. These catalysts are likewise generally separated from the polyether alcohols obtained after the synthesis, as a rule by deep bed filtration as described in DE-A 41 15 149 or DE-A 40 34 305.

A further way of preparing polyether alcohols by ring-opening polymerization of epoxides involves the use of multimetal cyanide catalysts, also referred to as DMC catalysts, preferably zinc hexacyanometalates. On this subject, reference may be made to DE-A 199 57 105.8, DE-A 198 40 846.3 and WO 98/44022 and the prior art cited in each of these.

Furthermore, a plurality of methods for separating the multimetal cyanide catalysts from the polyether alcohols are described in the prior art, and on this subject reference may once again be made to DE-A 199 57 105.8 and the prior art comprehensively cited therein.

The abovementioned DE-A 199 57 105.8 relates to a process for working up polyether alcohols in which the multimetal cyanide compound used as catalyst is removed from the polyether alcohol after the ring-opening polymerization by sedimentation. In this process, it is possible to recover the catalyst in chemically un-changed form from the polyether alcohol and the catalyst can then be reused in the preparation of polyether alcohols. On the other hand, the publications cited as prior art in DE-A-199 57 105.8 each relate to processes in which the DMC catalyst used is obtained in a form in which it cannot be reused or else the DMC catalyst is destroyed.

It is an object of the present invention to provide a further process for working up polyether alcohols which makes it possible to separate off the heterogeneous catalyst used therein without great expense, in particular in terms of apparatus, and in which this catalyst can be reused for the preparation of polyether alcohols. This is of particularly great economic importance for the DMC catalysts which are preferably used, since these catalysts are very expensive to produce. We have found that this object and other objects are achieved by the process of the present invention.

The present invention accordingly provides a process for working up polyether alcohols which have been prepared by ring-opening polymerization of epoxides by means of OH-functional starters using at least one heterogeneous catalyst, wherein the at least one heterogeneous catalyst is separated off from the polyether alcohol by means of a membrane separation process.

The present invention additionally provides a process for preparing polyether alcohols by ring-opening polymerization of epoxides by means of OH-functional starters, which comprises a work-up step comprising a process for working up as provided by the present invention.

In the process of the present invention, the heterogeneous catalyst which has been separated off from the polyether alcohols can be freed of the latter. This can be achieved by washing, for example with water or an organic solvent. It can then be converted, e.g. by drying, into a form in which it can be used for preparing polyether alcohols. This can be achieved, for example, by dispersion in solvents. This variant is employed particularly when the catalyst which has been separated off is to be used for preparing a different polyether alcohols and contamination by adhering residues of the first polyether alcohols is to be avoided in the preparation of the other polyether alcohol.

However, it is also possible to reuse the heterogeneous catalyst directly, without further work-up, for preparing a polyether alcohols. This process variant is employed particularly when the catalyst is, after the work-up, to be used for preparing the same polyether alcohol or when slight contamination by the first polyether alcohol is unimportant in the preparation of a different polyether alcohol.

In the process of the present invention, the heterogeneous catalyst is preferably concentrated during the separation from the polyether alcohols and obtained as a concentrated suspension in the polyether alcohol. As mentioned above, this concentrated catalyst suspension can subsequently be reused for the synthesis of a polyether alcohol.

The work-up process of the present invention can be carried out continuously or batchwise, i.e. the product mixture obtained after the ring-opening polymerization can be passed either continuously or batchwise through the separation apparatus, with, as mentioned above, the catalyst preferably being concentrated during this work-up.

The part of the polyether alcohol which passes through the separation apparatus and is thus essentially free of the heterogeneous catalyst is referred to as “permeate”, while the remaining part of the polyether alcohol in which the solid is preferably concentrated is referred to as “retentate”.

Heterogeneous catalysts which can be used or separated off in the preparative or work-up processes of the present invention include both basic catalysts and catalysts selected from the group consisting of multimetal cyanide compounds (DMC catalysts).

Examples of basic catalysts are:

Alkaline earth metal hydroxides and oxides, hydrotalcite, basic clays and basic antimonates, as described, for example, in EP-A 1 002 821.

However, preference is given to using DMC catalysts since they are significantly more active in the polymerization of epoxides than all other known classes of heterogeneous catalysts and can therefore be used in very much lower concentrations. This has the advantage, particularly in the context of the work-up process of the present invention, that a higher concentration rate and a higher yield of polyether alcohol can be achieved at the same catalyst content at the end point of the concentration by the membrane separation process or, when a suspension comprising the catalyst which has been separated off is recirculated to the preparation of the polyether alcohol, the recirculation rate of the polyether monool previously obtained as product into this (new) preparation of the polyether alcohol is lower.

Furthermore, DMC catalysts display a phenomenon which is referred to as “differential catalysis” in the literature. What is meant by differential catalysis is that in the alkoxylation of a mixture of starters of various molar masses, the starters having the lowest molar masses are preferentially alkoxylated, so that the molar mass differences are leveled out during the course of the polymerization reaction. This property proves to be particularly useful in the work-up process of the present invention since the catalyst which has been separated off is, if it is reused, used as a suspension in the end product, i.e. the finished polyether alcohol having a high molar mass. Differential catalysis prevents the quality of the new polyether alcohol to be prepared from being impaired by recirculation of the catalyst in suspension in previously finished polyether alcohol to the preparation of new polyether alcohol.

The DMC catalysts which can be used or separated off in the preparative or work-up process of the present invention are subject to no restrictions. For details of these catalysts and their preparation, reference may once again be made to DE-A 199 57 105.8, DE-A 198 40 846.3 and WO 98/44022.

Particular preference is given to using the following DMC catalysts:

Double metal cyanide catalysts as described in DE-A 198 40 846.3 comprising a double cyanide complex of the formula (I)

M¹ₐ[M²(CN)_{b}(A)_{c}]_{d}.fM¹_{g}Xₙ.h(H₂O).eL.kP, (I)

where

M¹ is at least one metal ion selected from the group consisting of

Zn²⁺, Fe²⁺, Co³⁺, Ni²⁺, Mn²⁺, Co²⁺, Sn²⁺, Pb²⁺, Mo⁴⁺, Mo⁶⁺, Al³⁺, V⁴⁺, V⁵⁺, Sr²⁺, W⁴⁺, W⁶⁺, Cr²⁺, Cr³⁺, Cd²⁺,

M² is at least one metal ion selected from the group consisting of

Fe²⁺, Fe³⁺, Co²⁺, Co³⁺, Mn²⁺, Mn³⁺, V⁴⁺, V⁵⁺, Cr²⁺, Cr³⁺, Rh³⁺, Ru²⁺, Ir³⁺,

where M¹ and M² are different,

A is an anion selected from the group consisting of halide, hydroxide, sulfate, carbonate, cyanide, thiocyanate, isocyanate, cyanate, carboxylate, oxalate and nitrate,

X is an anion selected from the group consisting of halide, hydroxide, sulfate, carbonate, cyanide, thiocyanate, isocyanate, cyanate, carboxylate, oxalate and nitrate,

L is a water-miscible ligand selected from the group consisting of alcohols, aldehydes, ketones, ethers, polyethers, esters, ureas, amides, nitriles and sulfides,

k is a fraction or integer greater than or equal to zero, and

P is an organic additive,

where a, b, c, d, g and n are selected so that the compound is electrically neutral,

e is the number of ligands coordinated and is greater than or equal to 0,

f is a fraction or integer greater than or equal to 0 and

h is a fraction or integer greater than or equal to 0.

Examples of organic additives are:

polyethers, polyesters, polycarbonates, polyalkylene glycol sorbitan esters, polyalkylene glycol glycidyl ethers, polyacrylamide, poly(acrylamide-co-acrylic acid), polyacrylic acid, poly(acrylamide-co-maleic acid), polyacrylonitrile, polyalkyl acrylates, polyalkyl methacrylates, poly(vinyl methyl ether), poly(vinyl ethyl ether), polyvinyl acetate, polyvinyl alcohol, poly-N-vinylpyrrolidone, poly(N-vinylpyrrolidone-co-acrylic acid), poly(vinyl methyl ketone), poly(4-vinylphenol), poly(acrylic acid-co-styrene), oxazoline polymers, polyalkylenimines, maleic acid and maleic anhydride copolymers, hydroxyethylcellulose, polyacetates, ionic surface-active and interface-active compounds, gallic acid or its salts, esters or amides, carboxylic esters of polyhydric alcohols and glycosides.

The polyether alcohols which can be worked up or prepared according to the present invention, i.e. polyether monools and polyether polyols and mixtures thereof, are subject to no restrictions. Preference is given to polyether alcohol of the formula (II):

R—[O-(AO)ₙ—H]ₘ (II),

where R is an alkyl, aryl, aralkyl or alkylaryl radical having from 1 to 60, preferably from 1 to 24, carbon atoms, AO are one or more, preferably from 1 to 3, C₂-C₁₀-alkylene oxides and n is an integer greater than or equal to 1, preferably from 1 to 1000 and m is an integer from 1 to 8. For polyether monools n is preferably 1 to 100, more preferably from 1 to 50, an m is 1. For polyether polyols n is preferably 1 to 1000, more preferably 1 to 500, and m is 2 to 8, more preferably 2 to3.

As compounds from which the radical R is derived, preference is given to alcohols and polyalcohols which have from 1 to 60 carbon atoms and may bear further functional groups which are not reactive toward alkylene oxides, e.g. ester groups. More preferred are alcohols having from 6 to 24 carbon atoms, particularly preferably from 9 to 20 carbon atoms. Such alcohols may be saturated, e.g. methanol, butanol, dodecanol or tridecanol, ethylen glycol, 1,2-propylene glycol, 1,3-propylene glycol, butanediol, pentanediol, hexanediol, glycerol, trimethylolpropane, pentaerythrol, glucose, or unsaturated, e.g. butenol, vinyl alcohol, allyl alcohol, diterpene alcohols such as geraniol, linalool or citronellol. It is also possible to use aromatic alcohols, preferably ones which are substituted by C₄-C₁₅-alkyl groups, e.g. phenol or nonylphenol. Further examples of alcohols which can be used are hexanol, heptanol, octanol, decanol, undecanol, tetradecanol, pentadecanol, hexadecanol, heptadecanol, octadecanol, nonadecanol, hexenol, heptenol, octenol, nonenol, decenol and undecenol.

Hydroxyalkyl esters of saturated and unsaturated acids such as (meth)acrylic acid, for example hydroxyethyl (meth)acrylate, are also possible.

In addition, it is also possible to use mixtures of such alcohols, in particular C₁₂-C₁₅-aliphatic alcohols.

It is preferred to use water, ethylen glycol, 1,2-propylene glycol, 1,3-propylene glycol, butanediol, pentanediol, hexanediol, glycerol, trimethylolpropane, pentaerythrol and glucose as starter for the polymerization process.

The alkylene oxides used are also subject to no restrictions, as long as they meet the abovementioned conditions. Preference is given to using ethylene oxide, propylene oxide, butylene oxide, pentene oxide, hexene oxide, cyclohexene oxide or mixtures thereof Particular preference is given to using propylene oxide.

The kinematic viscosity (Ubbelohde, 40° C.) of the polyether monools under consideration here is preferably less than 3000 mm²/s, more preferably less than 2000 mm2/s and in particular less than 1000 mm2/s.

The dynamic viscosity of the polyether polyols under consideration here is preferably less than 3000 mPas, more preferably less than 2000 mPas and in particular less than 1000 mPas.

The amount of heterogeneous catalyst present in the polyether alcohol before the catalyst is separated off varies depending on the class of catalyst used. When heterogeneous basic catalysts are used, they are present in the polyether alcohol obtained as product in amounts of from 0.05 to 3% by weight. When DMC catalysts are used, this content is in the range from 10 to 200 ppm.

In the process of the present invention, the heterogeneous catalyst is separated off from polyether alcohols by means of a membrane separation process. As membrane separation processes, preference is given to using microfiltration or cross-flow filtration or ultrafiltration.

In the membrane separation process used according to the present invention, part of the polyether alcohol is preferably taken off as permeate and the solid is concentrated in the remaining polyether alcohol (retentate). The permeate obtained is essentially free of the heterogeneous catalyst. This permeate preferably contains less than 20 ppm of metal, particularly preferably less than 10 ppm of metal, very particularly preferably less than 5 ppm of metal, in each case based on the total mass of permeate.

The amount of heterogeneous catalyst present in the retentate is in the range from 0.5 to 10% by weight, preferably from 2 to 8% by weight. The concentration factor at a catalyst concentration of 0.05% in the polyether alcohol after the synthesis and a concentration of 0.5% in the retentate is 10 and at a concentration of 10% by weight in the retentate is 200.

In the case of multimetal cyanide catalysts, the multimetal cyanide content of the retentate is from 0.5 to 10% by weight, preferably from 2 to 8% by weight. The concentration factor at a catalyst concentration of 100 ppm in the polyether ex synthesis and a concentration of 0.5% in the retentate is 50, and at a concentration of 10% by weight in the retentate is 1000.

The optimum solids content in the retentate is determined by various boundary conditions, depending on the polyether alcohol and the heterogeneous catalyst used. One of these boundary conditions is, for example, that the retentate should generally remain pumpable. Moreover, very high solids content in the retentate can have an adverse effect on the permeate performance of the membrane process.

To separate off the heterogeneous catalyst, the synthesis mixture comprising the polyether alcohol is brought into contact with a membrane under superatmospheric pressure and a permeate is taken off under atmospheric pressure from the reverse side of the membrane. A catalyst concentrate (retentate) and a virtually catalyst-free permeate are obtained. To avoid appreciable buildup of a covering layer of catalyst on the surface of the membrane, which leads to a significant decrease in the permeate flux, a relative velocity between membrane and suspension of 0.5-10 m/s is generated by pumped circulation, mechanical movement of the membrane or stirrers between the membranes. Concentration can be achieved in a batch mode by passing the synthesis mixture a number of times through the membrane module or continuously by means of a single pass through one or more feed and bleed stages connected in series. The concentrated catalyst suspension in polyether alcohol obtained in this way can subsequently be reused for the synthesis of polyether alcohol.

The membrane process of microfiltration or crossflow filtration is carried out using membrane separation layers having pore diameters of from 5000 nm to 100 nm, while the membrane process of ultrafiltration is carried out using membrane separation layers having pore diameters of from 100 nm to 5 nm. The membranes can be used in flat, tubular, multichannel, capillary or wound geometries, for which appropriate pressure housings which allow separation between catalyst suspensions and the permeate (filtrate) are available. The transmembrane pressures between retentate and permeate to be applied are dependent essentially on the diameter of the membrane pores and the mechanical stability of the membrane at the operating temperature and are, depending on the membrane type, from 0.5 to 60 bar. The operating temperature depends on the product stability and the membrane stability. The permeate fluxes increase drastically with an increase in temperatures. Temperatures of, for example, up to 140° C. can be achieved when using ceramic membranes.

The separation layers can comprise organic polymers, ceramic, metal or carbon. For mechanical reasons, the separation layers are generally applied to a singlelayer or multilayer substrate made of the same material as the material of the separation layer or of one or more different materials. Examples are:

| Separation layer | Substrate (coarser than separation layer) |
|---|---|
| Metal | Metal |
| Ceramic | Metal, ceramic or carbon |
| Polymer | Polymer, metal, ceramic or ceramic on metal |
| Carbon | Carbon, metal or ceramic |

Ceramic: e.g. α-Al₂O₃, γ-Al₂O₃, ZrO₂,
TiO₂ SiC, mixed ceramic materials
Polymer: e.g. PTFE, PVDF, polysulphone

Since high temperatures are advantageous because of the associated higher permeate fluxes, the use of purely inorganic membranes (including carbon) is preferred.

The process of the present invention for preparing polyether alcohols comprising polyether monools and polyether polyols comprises the work-up process of the present invention as one step. The actual preparation of the polyether alcohols is carried out by continuous or batchwise processes known from the prior art, with preference being given to the continuous mode of operation. In this respect, reference may be made to the prior art cited at the outset, namely DE-A 199 57 105.8, DE-A 198 40 846.3 and WO 98/44022 and the publications cited therein.

In particular, the present invention provides a process for preparing polyether alcohols in which the catalyst which has been worked up according to the present invention is used either as such or in the form of a suspension in one or more polyether alcohols as catalyst in a subsequent preparation of polyether alcohols, i.e. is recirculated to the preparative process after work-up. Such a process has particularly good economics.

The polyether alcohols under consideration here are used, for example, as carrier oils in fuel additive mixtures, in which case it is very advantageous to remove the catalyst employed from the polyether alcohol obtained as reaction product. This applies particularly to the polyether alcohols used as carrier oils, since catalyst residues can firstly have a corrosive action and, secondly, can lead to undesirable deposits and emissions on combustion in the engine. They are also used in the automobile industry as fuel additives, in brake fluids and also as surface-active substances or solvents.

The invention is illustrated by the following examples.

### EXAMPLES

### Preparative Example 1

### Preparation of Hexacyanocobaltic Acid

71 of strong acid ion exchanger in the sodium form (Amberlite 252 Na, Rohm&Haas) were placed in an ion exchange column (length: 1 m, volume: 7.7 1). The ion exchanger was subsequently converted into the H form by passing 10% strength hydrochloric acid through the ion exchange column at a rate of 2 bed volumes per hour for 9 hours until the Na content of the output was less than 1 ppm. The ion exchanger was subsequently washed with water until neutral.

The regenerated ion exchanger was then used to prepare an essentially alkali-free hexacyanocobaltic acid. For this purpose, a 0.24 molar solution of potassium hexacyanocobaltate in water was passed over the exchanger at a rate of 1 bed volume per hour. After 2.5 bed volumes, the potassium hexacyanocobaltate solution was replaced by water. The 2.5 bed volumes obtained had an average hexacyanocobaltic acid content of 4.5% by weight and an average alkali metal content of less than 1 ppm. The hexacyanocobaltic acid solutions used in the further examples were diluted appropriately with water.

### Preparative Example 2

### Preparation of a Suspension of the Multi Metal Cyanide Compound from Preparative Example 1 in Tridekanol N

1600 g of aqueous hexacyanocobaltic acid (cobalt content: 9 g of cobalt/l) were placed in a stirred vessel having a volume of 30 1 and fitted with a disk stirrer, immersed tube for introduction of reactants, pH probe and scattered light probe and the initial charge was heated to 50° C. while stirring. While stirring (stirrer power: 1 W/l), 9224 g of aqueous zinc acetate dihydrate solution (zinc content: 2.6% by weight) which had likewise been heated to 50° C. was subsequently fed in over a period of 15 minutes.

351 g of Pluronic PE 6200 (BASF AG) were subsequently added while stirring. 3690 g of aqueous zinc acetate dihydrate solution (zinc content: 2.6% by weight) were then metered in at 50° C. over a period of 5 min while stirring (stirring energy: 1 W/l). The suspension was stirred at 50° C. until the pH had dropped from 4.0 to 3.2 and remained constant. The precipitation suspension obtained in this way was subsequently filtered and the precipitate was washed on the filter using 6 times the cake volume of water.

The moist filter cake was subsequently dispersed in 20 g of Tridekanol N (BASF AG) in a stirred apparatus having a volume of 30 l and dewatered under reduced pressure at 80° C.

The resulting suspension of the multimetal cyanide compound in Tridekanol N was subsequently redispersed by means of a slotted rotor mill. The suspension obtained had a multimetal cyanide content of 5% by weight.

### Preparative Example 3

### Preparation of a Polyether Monool as Carrier Oil by Means of Multi Metal Cyanide Catalysis from Preparation Example 2

100.00 kg of Tridekanol N were introduced into a stirred reactor provided with an external heat exchanger and having a volume of 600 l, and 2.14 kg of a 5% strength multimetal cyanide suspension in Tridekanol N, corresponding to 200 ppm of catalyst based on the final amount, were added. The mixture was subsequently dewatered under reduced pressure (<50 mbar) at 115° C. to a water content of <0.02%. The vacuum was broken while introducing nitrogen.

After a subsequent pressure test, the mixture was heated to 135° C. at an initial pressure sure of 0.5 bar. For monitoring of the reaction (commencement of reaction), about 24 kg of propylene oxide were introduced at 135° C., and this reacted with a decrease in pressure after an induction period of about 6 minutes. The remaining 411.3 kg of the amount of propylene oxide required (total amount: 435.2 kg of PO) was subsequently fed in according to PO metering ramps at 135° C. over a period of about 3 hours at a maximum pressure of about 2 bar. The reaction mixture was subsequently cooled to 80° C., depressurized under nitrogen and stripped under reduced pressure (<30 mbar) for about 60 minutes.

The work-up of the carrier oil obtained in this way by means of crossflow filtration is described in detail in example 1 below.

### Example 1

### Separation of the Multi Metal Cyanide Compound from Preparative Example 3 by Means of Membrane Filtration

The synthesis suspension obtained, containing 200 ppm of catalyst, was concentrated to about 3.5% in a batch process. A ceramic membrane having a separation limit (pore diameter) of 50 nm was used. The separation layer comprised ZrO₂ which had been applied to a multilayer substrate of alpha-Al₂O₃ on the inside of round channels having a diameter of 6 mm. The suspension was pumped through the membrane channels at 2 m/s via a circulation vessel. The process temperature was 70° C. and the transmembrane pressure was 3 bar. The Co and Zn content determined in the permeate was <1 ppm at the beginning and at the end of the concentration process.

Physicochemical data (after membrane filtration):

| | | |
|---|---|---|
| Kinematic viscosity at 40° C.: | 56.5 | mm²/s |
| Residual metal content: | <1 | ppm |
| Hydroxyl number: | 52 | mg KOH/g |
| Kaufmann iodine number (double | 0.1 | g iodine/100 g |
| bond content): | | |
| Propylene oxide content: | 1.8 | ppm |
| Water content: | 0.02 | |
| Density at 20° C.: | 0.9658 | g/cm³ |

### Example 2

### Reuse of the Crossflow-Filtered Catalyst from Example 1

200 g of Tridekanol N and 6.11 g of the above-described multimetal cyanide catalyst suspension obtained from the crossflow filtration (about 3.5% strength) were placed in a stirred reactor having a volume of about 2 l. The amount of catalyst suspension corresponded to 200 ppm of multimetal cyanide based on the final amounts. The mixture was subsequently heated to 120° C. and dewatered at this temperature for 2 hours to a water content of <0.05%. The vacuum was broken with introduction of nitrogen. After a subsequent pressure test, the mixture was heated to 135° C. at an initial pressure of 0.5 bar. For monitoring of the reaction (commencement of reaction), about 50 g of propylene oxide were introduced at 135° C., and this reacted with a decrease in pressure after an induction period of about 6 minutes. The remaining 820 g of the amount of propylene oxide required (total amount: 870 g of PO) was subsequently fed in according to PO metering ramps at 135° C. over a period of about 3 hours at a maximum pressure of about 8 bar. The mixture was subsequently stirred at 135° C. for another 2 hours, depressurized under nitrogen and the reaction mixture was stripped in a laboratory rotary evaporator at about 100° C. and a pressure of <30 mbar, and subsequently filtered through a deep bed.

Physicochemical data:

| Kinematic viscosity at 40° C.: | 60 | mm²/s |
|---|---|---|
| Residual metal content: | Co < 2 ppm, Zn 4 ppm | |
| Hydroxyl number: | 51 | mg KOH/g |
| Kaufmann iodine number (double | <0.1 | g iodine/100 g |
| bond content): | | |
| Propylene oxide content: | <1 | ppm |
| Water content: | 0.07% | |
| Density at 20° C.: | 0.9677 | g/cm³ |

### Preparative Example 4

### Preparation of a Suspension of the Multimetal Cyanide Compound from Preparative Example 1 in a Glycerol Propoxylate Polymer

370 kg of aqueous hexacyanocobaltic acid (cobalt content: 9 g of cobalt/l) were placed in a stirred vessel having a volume of 800 l and fitted with a disk stirrer, immersed tube for introduction of reactants, pH probe and scattered light probe and the initial charge was heated to 50° C. while stirring. While stirring (stirrer power: 1 W/l), 209.5 kg g of aqueous zinc acetate dihydrate solution (zinc content: 2.7% by weight) which had likewise been heated to 50° C. was subsequently fed in over a period of 50 minutes.

8 kg of Pluronic PE 6200 (BASF AG) and 10.7 kg water were subsequently added while stirring. 67.5 kg of aqueous zinc acetate dihydrate solution (zinc content: 2.6% by weight) were then metered in at 50° C. over a period of 20 min while stirring (stirring energy: 1 W/l).

The suspension was stirred at 50° C. until the pH had dropped from 3.7 to 2.7 and remained constant. The precipitation suspension obtained in this way was subsequently filtered and the precipitate was washed on the filter using 400 l of water.

The moist filter cake was subsequently dispersed by means of a slotted rotor mill in a glycerol propoxylate polymer (molecular weight 900 g/mol), having active hydrogen atoms. The suspension obtained had a multimetal cyanide content of 5% by weight.

### Preparative Example 5

### Preparation of a Polyether Polyol by Means of the Multimetall Cyanid Catalyst from Preparative Example 4

2.00 kg of a propoxylated glycerol having a molecular weight of 400 g/Mol and 0.196 kg of a propoxylated ethylene glycol having a molecular weight of 250 g/mol were introduced into a stirred reactor having a volume of 20 l, and 38 g kg of a multimetal cyanide suspension from preparative example 4 were added. The mixture was subsequently dewatered under reduced pressure (<50 mbar) at 110° C. in vacuum. After the vessel was set under pressure with nitrogen and subsequently within 3.5 h 3,45 kg propylene oxide and 1.9 kg ethylene oxide were metered. After 2.0 kg propylene oxide were metered. The content of the reactor was stirred for 0.6 h, followed by degassing at 115° C. and 9 mbar.

Physicochemical data:

| | | |
|---|---|---|
| Hydroxyl number: | 47.4 | mg KOH/g |
| Dynamic viscosity at 25° C.: | 578 | mPas |

| | |
|---|---|
| Residual metal content: | Co = ppm, Zn = 27 ppm |

After filtration a polymer had been obtained with a content of Zn and Cd being beyond the detection limit.

| | | |
|---|---|---|
| Hydroxyl number: | 47.4 | mg KOH/g |
| Dynamic viscosity at 25° C.: | 578 | mPas |

### Example 3

### Example 3

### Separation of the Multi Metal Cyanide Compound from Preparative Example 5 by Means of Membrane Filtration

The synthesis suspension obtained, containing 100 ppm of catalyst, was concentrated to about 3% in a batch process. A ceramic membrane having a separation limit (pore diameter) of 50 um was used. The separation layer comprised ZrO₂ which had been applied to a multilayer substrate of alpha-Al₂O₃ on the inside of round channels having a diameter of 6 mm. The suspension was pumped through the membrane channels at 2 m/s via a circulation vessel. The process temperature was 70° C. and the transmembrane pressure was 3 bar. The Co and Zn content determined in the permeate was <1 ppm at the beginning and at the end of the concentration process.

### Example 4

### Reuse of the Crossflow-Filtered Catalyst from Example 3

The synthesis was run in a purified and dry and stirred reactor having a volume of 20 l. 2.00 kg of a propoxylated glycerol having a molecular weight of 400 g/Mol and 0.196 kg of a propoxylated ethylene glycol having a molecular weight of 250 g/mol were introduced into the reactor and treated with 64 g of the filtered multimetal cyanide suspension. The amount of catalyst suspension corresponded to 100 ppm of multimetal cyanide based on the final amounts. The reactor was rendered inert with nitrogen and dewatered for 1.5 h in vacuum. After the vacuum was broken with introduction of nitrogen the temperature was increased to 115° C. Subsequently 3.45 kg propylene oxide were metered followed by a mixture of 10.2 kg propylene oxide and 1.9 kg ethylene oxide. Subsequently another portion of 2.0 kg propylene oxide were added. After stirring for 0.6 h the reactor was degassed under 9 mbar. The resulting polyether polyol could be characterized by following datas:

| | |
|---|---|
| Hydroxyl number: | 47.2 mg KOH/g |
| Dynamic viscosity at 25° C.: | 605 mPas |
| Residual metal content: | Co = 12 ppm |
| | Zn = 27 ppm |

After filtration a polymer had been obtained with a content of Zn and Cd being beyond the detection limit.

| | |
|---|---|
| Hydroxyl number: | 47.2 mg KOH/g |
| Dynamic viscosity at 25° C.: | 605 mPas |

The content of metal in examples 1 to 4 was determined by means of flame-emission spectroscopy and the method of inductive coupled plasma (JCP).

The kinematic viscosity of examples 1 and 2 has been measured by means of a viscometer according to Ubbelohde and DIN 51562.

The kinematic viscosity of examples 1 and 2 has been measured by means of a rotary viscometer with plate and cone (Rheolab-MC-1 of company Physica) according to DIN 53018 and 53019.

We claim:

1. A process for working up polyether alcohols, which have been prepared by ring-opening polymerization of epoxides by means of OH-functional starters using at least one heterogeneous catalyst, wherein the at least one heterogeneous catalyst is separated off from the polyether alcohol by means of a membrane separation process.

2. A process as claimed in claim 1, wherein the heterogeneous catalyst is not changed chemically after the ring-opening polymerization.

3. A process as claimed in claim 1 or 2, wherein the membrane separation process is selected from the group consisting of microfiltration, crossflow filtration and ultrafiltration.

4. A process as claimed in any of claims 1 to 3, wherein the membranes used in the membrane separation process have separation layers whose pore diameter is from 5 to 5000 nm.

5. A process as claimed in any of claims 1 to 4, wherein the separation layers of the membranes used in the membrane separation process comprise organic polymers, a ceramic material, metal or carbon or a combination of two or more thereof.

6. A process as claimed in any of claims 1 to 5, wherein the separation of the heterogeneous catalyst from the polyether alcohol is carried out continuously or batchwise.

7. A process as claimed in any of claims 1 to 6, wherein the heterogeneous catalyst is a multimetal cyanide catalyst.

8. A process for preparing polyether alcohols by ring-opening polymerization of epoxides by means of OH-functional starters, which comprises a work-up step comprising a process as claimed in any of claims 1 to 7.
